(19) United States
(12) Patent Application Publication
(10) Pub. No.: US 2003/0147977 A1
(43) Pub. Date: Aug. 7, 2003

(54) Topical preparation for treating acne and hirsutism

(76) Inventor: David S. Goodman, Naples, FL (US)

Correspondence Address: Riker, Danzig, Scherer, Hyland & Perretti LLP, Headquarters Plaza, One Speedwell Avenue, Morristown, NJ, 07962, US

(21) Appl. No.: 10/345,896

(22) Filed: Jan. 16, 2003

Related U.S. Application Data
(63) Continuation-in-part of application No. 10/097,001, filed on Mar. 13, 2002.
Application No. 10/097,001 is a continuation-in-part of application No. 09/563,555, filed on May 3, 2000, now Pat. No. 6,358,541.

(51) Int. Cl. A61K 35/78

(52) U.S. Cl. 424/727; 424/401

(57) ABSTRACT

An improved method and preparation for the treatment of acne and hirsutism comprises topically applying an effective amount of a saw palmetto berry extract, in combination with two classes of low irritability penetrating agents that enhance penetration of the extract into hair follicles and sebaceous glands. The low irritability penetration agents are selected from the group (A) comedolytics consisting of adapalene, tretinoin, tretinoin gel microsponges, retinaldehyde, retinol, and tazarotene, and (B) keratinolytics consisting of the beta hydroxy acid, salicylic acid, and the alpha hydroxy acid, glycolic acid. Polyolprepolymer-2 may be incorporated into the preparation as well.

### RELATED APPLICATIONS

This application is a Continuation-in-Part of U.S. Ser. No. 10/097,001 filed Mar. 13, 2002 which in turn is a continuation in part of Ser. No. 09/563,555 filed May 3, 2000 both or the instant inventor.

### BACKGROUND OF THE INVENTION

This invention relates to an improved preparation and method for treating both acne and hirsutism, or unwanted facial and body hair in women; and more particularly to a preparation comprising a topically active extract of saw palmetto berries, in combination with two classes of penetration enhancing agents included to enhance penetration of the extract into hair follicles and sebaceous glands.

Pathogenens of Acne

Acne is caused by a complex interaction of excessive sebum production, colonization of hair follicles by Proprionibacterium acnes, and follicular plugging or comedone formation.

Sebum is the oily secretion of sebaceous glands. These glands drain directly into hair follicles and are present mainly on the face, chest and back. Sebum production is predominantly controlled by androgens manufactured in the testes, adrenal glands, and ovaries. High levels of androgens are associated with increased sebum production.

Dihydrotestosterone (hereinafter referenced to as DHT), is the prime androgen responsible for excessive sebum production. Skin affected by acne contains two to thirty times more DHT than normal skin. Much of this excess of DHT is due to conversion of testosterone into DHT directly in acne prone skin. This conversion is mediated by the enzyme 5-alpha reductase.

Follicular plugs consist of collections of sebum, keratinocytes, and keratin. They occlude the follicular orifice and prevent the further drainage of the contents of the follicle. The greater the quantity of sebum, the more likely that a follicular plug will form. The clogged follicle is known as a comedone. Sebum production into a comedone or clogged follicle, as well as the proliferation of Proprionibacterium acne, a bacteria which commonly colonizes hair follicles ultimately leads to the rupture of the wall of the comedone and allows the spilling of sebum and bacteria into the surrounding skin. Thus leads to the formation of inflammatory acne lesions.

Saw palmetto, (serenoa repens), is a small berry-bearing palm native to the southeast United States. Saw palmetto berry extract, (hereinafter referred to as SPBE) has been shown to block 5-alpha reductase, the enzyme that converts the hormone testosterone into dihydrotestosterone. Once again, this is the major androgen implicated in stimulating sebum production. SPBE also blocks the binding of DHT to androgen receptors. Thus, in treating acne SPBE may act either by blocking the formation of DHT, by inhibiting binding of DHT to the androgen receptors, or both.

A need exists for a SPBE formulation and method for effectively treating acne. Especially needed is a SPBE topical formulation that offers efficacy and high penetration into hair follicles and sebaceous glands, while causing minimal irritation.

Acne Treatments

Current accepted acne therapies include both topical and systemic products. Topical products include:

(a) antibiotics such as erythromycin, clindamycin and benzyl peroxide which decrease the number of Proprionibacterium acnes in follicles, or

(b) keratinolytics such as salicylic acid and glycolic acid which help remove keratin from the surface of the skin and thereby prevent comedone formation, or

(c) comedolytics such tretinoin, adapalene and tazarotene which decrease keratinocyte proliferation at follicular openings and thereby also help prevent comedone formation.

Hirsutism

Hirsutism is the abnormal growth of large terminal hairs in women in androgen sensitive areas. Most often these areas include the mustache, beard and chin areas, as well as the pubic escutcheon and lower abdomen. Unwanted hair may also be present on the thighs and trunk as well. Terminal hair growth in these areas is usually due to excess androgenic stimulation of the hair follicles.

Idiopathic hirsutism is the most common type of hirsutism. It is felt to be due to increased conversion of testosterone into its more active form, dihydrotestosterone or DHT. This conversion occurs in peripheral tissues close to the hair follicles, and it is mediated by the enzyme 5 alpha reductase.

Hirsutism may also be caused by conditions in which an excess of androgens are synthesized by the ovaries, adrenal glands or tumors.

Treatment of hirsutism depends in part on its cause. A prudent search for a source of excess androgen production is important to rule out androgen secreting tumors, ovarian disease and other systemic hormonal abnormalities. In the setting of normal menstruation and normal hormone levels, therapy for idiopathic hirsutism can be instituted.

Readily available non-medical therapies for idiopathic hirsutism include the plucking of hairs, waxing, electrolysis, and depilatories. Medical therapies include various antiandrogenic agents—such as synthetic progestins, dexamethesone, gonadotropin releasing hormone agonists, ketoconazole, spironolactone, oral contraceptives, and the antiandrogens cyproterone acetate and flutamide. The topical agent Vaniqa has also been used. However, unfortunately, none of the above therapies is free of undesirable side effects.

As noted previously, SPBE has been shown to block 5 alpha reductase, the enzyme that converts testosterone into dihyrotestosterone or DHT. Saw palmetto berry extract also blocks the binding of DHT to androgen receptors. Thus, in treating hirsutism, SPBE is an effective agent and may act either by blocking the formation of DHT, or by inhibiting binding of DHT to the androgen receptors, or both without undue side effects.

### PRIOR ART

U.S. Pat. No. 6,039,950 presents an extensive disclosure for making pharmaceutical grade saw palmetto materials. The possible use of SPBE in oral compositions for treating acne and hirsuitism is noted as a possible future use. (col 7 line 66 - col 8 line 3). No actual data is presented. Numerous possible uses for the medicament, Permixon, a purified form of SPPE, are suggested.

U.S. Pat. No. 6,117,429 deals with reducing potential adverse effects of androgenic testosterone precursors by interfering with the production or the action of testosterone and estrogen metabolites by use of nutrient combinations. Such precursors enhance hormone responsive illness such as hirsuitism or acne in women as an undesirable side effect. Saw palmetto berry is listed as one of many natural agents which can be included in their compositions to prevent the side effects caused by testosterone precursors.

In both U.S. Pat. No. 6,039,950 and U.S. Pat. No. 6,117,429, the saw palmetto berry composition is taken orally rather than comprising a topical preparation pursuant to the present invention.

In U.S. Pat. No. 6,225,299 Gibbs et al. describe the use of gestogen and dienogest as antiandrogens to decrease sebum production when used topically to control acne. They do not describe the use of SPBE nor do they incorporate any penetration-enhancing agents into their topical products. In fact the preparations they describe contain potentially comedogenic or acne causing substances such as peanut oil, castor oil, beeswax, hardwax, lanolin, and paraffin.

In U.S. Pat. No. 6,174,892. Gormley, et al. describe the treatment of acne with oral finasteride, a known 5 alpha reductase inhibitor. They do not discuss the use of SPBE.

Fauran (FR2556968A1) and Fauran (EP0204877a1) describe a composition for the topical treatment of acne containing a hexane extract of saw palmetto in a base with other agents that assures penetration of the active ingredients into the skin.

### BRIEF DESCRIPTION OF THE INVENTION

The invention provides an improved preparation and a method for treating acne and hirsuitism. A topically applied SPBE formulation is hereby taught which offers efficacy and high penetration into the hair follicles and sebaceous glands while causing minimal irritation and side effects.

The preparation comprises an extract of saw palmetto berries preferably containing phytosterols and a member of both the keratinolytic and comedolytic classes of penetration agents to enhance the penetration of the extract into the follicles and sebaceous glands. More specifically, the improved low irritability topical preparation comprises saw palmetto berry extract containing phytosterols and one or more penetration enhancing constituents selected from the group of comedolytic agents consisting of adapalene, tretinoin, tretinoin gel microsponges, retinaldehyde, retinol and tazarotene; and a second penetration enhancing agent selected from the class of keratinolytic agents consisting of salicylic acid, and glycolic acid.

In another embodiment, the improved low irritability topical preparation comprises an active saw palmetto berry extract containing phytosterols, polyolprepolymer-2 and the aforesaid penetration enhancing constituents selected from both the keratinolytic and comedolytic classes of penetrating agents.

The preparations of the invention may include a vehicle suitable for topical application to the face and/or trunk in the form of a liquid, a gel, a foam, a cream, a lotion, a cleanser, or a pad dampened with a liquid.

The method for the prevention of acne or hirsuitism comprises applying to the affected areas a low irritability preparation comprising a combination of an effective amount of a saw palmetto berry extract containing phytosterols, and a penetration enhancing constituents selected from the group consisting of (A) adapalene, tretinoin, tretinoin gel microsponges, retinaldehyde, retinol, and tazarotene, and (B) a second penetration enhancing constituent selected from salicylic acid and glycolic acid. In another embodiment, the method comprises applying to affected areas a low irritability preparation comprising saw palmetto berry extract containing phytosterols, polyolprepolymer-2 and penetration enhancing constituents selected from the aforesaid groups (A) and (B).

Keratinolytics are agents that act to remove keratin or compact dead cells from the surface of the skin. In doing this, they also remove the keratin that overlies the pores, follicles and comedones of the skin. By exposing the pores and follicles, they allow for externally applied agents to more easily penetrate into those pores and follicles. Thus, they are penetration enhancing agents.

The keratinolytics employed in this invention include the alpha hydroxy acid, glycolic acid, and the beta hydroxy acid, salicylic acid.

During the course of normal skin function pores and follicles become occluded or blocked by cells growing along the walls of the pores, and by the secretion of sebum into the pores by sebaceous glands. Occluded follicles are called comedones. Comedolytic agents are substances that act to open comedones by removing the plugs of cells and sebum that occlude the pores. By unplugging pores and follicles, they also allow for externally applied agents to more easily penetrate into these pores and follicles. While the mechanism of action of comedolytics in opening pores is different than that of keratinolytics, the comedolytics are also penetrtion enhancing agents.

The comedolytics used in the present invention include retinol, retinaldehyde, tretinoin, tretinoin gel microsponges, adapalene, and tazarotene.

By employing both a keratinolytic and a comedolytic agent in this invention, two penetration enhancing substances that work via different but complementary mechanisms are utilized. First the keratinolytic peels away the dead cells that cover the skin. They thus expose the underlying pores, comedones, and follicles. The comedolytic then removes these plugs of cells and sebum from the follicles. The use of a keratinolytic and a comedolytic serves to enhance the penetration of SPBE into pores and follicles, allowing the SPBE to exert its maximum effect on sebaceous glands and hair follicles.

The methods of applying the present SPBE compositions are described below.

### DETAILED DESCRIPTION OF THE INVENTION

The present invention provides low irritability topically applied formulations of SPBE offering enhanced effectiveness in treating and preventing acne and hirsuitism by their greater ability to penetrate the hair follicles and sebaceous glands of the face and trunk. In addition, there is provided by the invention a method for controlling acne and hirsutism by application to the face, trunk and other affected areas of a preparation comprised of the improved formulations.

The method of application of the SPBE formulation is critical to the effectiveness of the invention. Important variables include the vehicle chosen, and the frequency and duration of application of the product.

This invention is described by a number of formulations and vehicles. Each vehicle is suited for use on a different type of skin. Alcohol based solutions and pads should be used on oily skin. They will help to remove unwanted excess oil from the skin without adding additional moisture. These would be well suited for use on the face of persons with acne or a combination of acne and hirsutism. In contrast, a lotion or cream should be used on dry or less oily skin. These vehicles will serve to moisturize the skin and protect it from irritation. Gels are perhaps most suitable for oily skin, but are reasonable to use on all skin types. They are also ideal to use over large hair bearing areas as they rub in well. Gels are particularly appropriate for use on acne prone chests, shoulders and upper backs. Similarly, foams which volatilize easily are excellent to use on large hair bearing areas.

Initially, all product formulations should be applied once daily. After two weeks, in the absence of allergy or irritation, the frequency of application should be increased to twice daily. All formulations should be applied after washing with a mild soap, but before the use of moisturizers, sunscreens or makeups.

Improvement should be noted in the treatment of facial acne within approximately one month. Additional improvement should continue for four or more months. Responses on the trunk may take longer because of thicker skin in these areas and slower penetration of the products into hair follicles.

Responses in hirsutism will be slower than in acne because of the growth pattern of hairs. Only after the large unwanted terminal hairs that characterize hirsutism have completed their growth cycle and fallen out, will meaningful improvement be seen. These large hairs will then be replaced by tiny, light, barely visible vellus hairs. Such improvement should be noted after four to six months.

Once a steady state has been readied and no additional improvements is noted in the acne or hirsutism, application of the invention can be decreased to once daily. This should occur after approximately four months in the case of acne and six to eight months in the treatment of hirsutism. In some instances, every other day therapy may prove adequate for a maintenance regimen.

This invention describes SPBE in conjunction with several penetration enhancing agents. Thus, using this invention will not only increase the penetration of SPBE into hair follicles, but it will increase the penetration of other topically applied agents as well. This should increase the effectiveness of these agents. It is critical that the invention be applied appropriately with respect to these other agents so as to maximize the effectiveness of all products used.

Topical medication for the treatment of acne include washes, pads, solutions, gels, lotions and creams. There is currently only one prescription provided for the treatment of hirsutism. It is known as Vaniqa, and it is a cream. This treatment does not act by interfering with the conversion of testosterone to DHT nor with the binding of DHT to androgen receptors. This medication should therefore work synergistically with the invention.

When used in a cream or lotion vehicle, this invention should be applied after the use of a wash, pad, solution or gel formulation of a different type of product.

When used as a gel or foam, it should be applied after washes, pads or solutions of another type of product, and before any other creams or lotions.

When used as a solution, the SPBE product should be used after washes or pads of another product, but before any other gels, lotions or creams.

SPBE pads should be used before solutions, gels, lotions or creams of another product. The pads, however, should not be used for a minimum of one hour after a wash or a pad formulation of another product has been used. Doing so may remove some of the active ingredient of that other product from the skin.

When used in a vehicle similar to the vehicle of another product, the SPBE product can be used before, with, or after that other product.

By way of summary, all product formulations are applied at least once daily after washing with mild soap, but before use of moisturizers, sunscreens or makeup. In the absence of allergy or irritation, the frequency of application is increased to twice daily. Improvement in facial acne typically occurs in one month. Improvement may continue for four or more months.

Amount Needed

A minimum concentration of purified SPBE of approximately 0.5 weight percent in the preparation is applied to the skin. This could be created, for example, by mixing 0.5 grams of 85%-95% purified SPBE with 99.5 grams of vehicle. Preferably, the concentration of SPBE should be 1.0 weight percent or higher. Most preferably, the concentration of SPBE should be 2.0 weight percent or higher.

In order to treat an area the size of a typical face, approximately 1 cc to 2 cc of solution or lotion, and 1 to 2 grams of gel or cream are required per application.

Extraction Method

The SPBE constituent of the present composition has been obtained from various commercial sources, including P.F. M\overline{e}dicaments, of Paris, France, the makers of Permixon®; the Saw Palmetto Harvesting Company in Frostproof Fla.; and U.S. Nutraceuticals, in Eustice, Fla.

It has now been found that particularly advantageous results are obtained by the use of Supercritical CO₂ extraction of the biologically active components of saw palmetto as performed by U.S. Nutraceuticals. An extract is obtained that is absolutely pure and solvent free. This is in contrast to the hexane and alcohol solvent extraction processes used by other manufacturers that provide a SPBE contaminated by significant amounts of solvent.

When carbon dioxide, (CO₂), is compressed above 73 bars at a temperature above 31° C. it is transformed into a dense gas known as Supercritical CO₂. Supercritical CO₂ has an extremely high solvating capacity. By manipulating the density and temperature of the Supercritical CO₂ and saw palmetto, Supercritical CO₂ extraction yields an extract that is extremely clean and solvent free. This process also allows extraction of more of the botanical's functional components. Ideally, for the preparation of saw palmetto berry extract, the pressures utilized in this process should be 500-700 bars, and the temperature should be 70-100° C.

U.S. Nutraceuticals of Eustice, Fla. currently uses the Supercritical CO₂ extraction process to prepare saw palmetto berry extract. With their “SuPure” process, they pass the subject materials and Supercritical CO₂ through a series of separators at successively lower pressures and temperatures to isolate the subject's biologically active components.

By using the Supercritical CO₂ extraction process in general, or the “SuPure” process of U.S. Nutraceuticals in particular, a saw palmetto berry extract is obtained that is solvent free for treating acne and/or hirsutism. When combined with the penetration enhancing agents, a clearly superior product is obtained.

In a preferred embodiment of the preparation of the present invention, the SPBE constituent is the super pure material sold by U.S. Nutraceuticals of Eustice, Fla. This SPBE is non-irritating when applied to the scalp. Typically, it has the following major components and concentrations:

| | |
|---|---|
| Phytosterols | |
| capesterol | about 0.01 to about 0.1 wt. % |
| beta-sitosterol | about 0.1 to about 0.4 wt. % |
| stimasterol | about 0.01 to about 0.1 wt. % |
| Total sterols | greater than about 0.15 wt. % |
| Fatty Acids | |
| caproic | about 1.0 to about 3.0 wt. % |
| caprylic | about 1.0 to about 3.0 wt. % |
| capric | about 1.0 to about 3.0 wt. % |
| lauric | about 25 to about 32 wt. % |
| cis-linoleic | about 3.0 to about 5.0 wt. % |
| linolenic | about 0.5 to about 2.0 wt. % |
| myristic | about 10 to about 15 wt. % |
| oleic | about 26 to about 35 wt. % |
| palmitic | about 7 to about 11 wt. % |
| stearic | about 1.0 to about 2.0 wt. % |

Other phytosterols, other fatty acids and other minor components may also be present without effecting the utility of the SPBE. The phytosterols are believed to be the active agents in the SPBE.

The preparation of the invention comprises the SPBE and at least one penetration enhancing constituents selected from each of groups (A) comedolytics consisting of adapalene, tretinoin, tretinoin gel microsponges, retinol, retinaldehyde, and tazarotene and group (B) keratinolytics consisting of salicylic acid, and glycolic acid. In another embodiment, the preparation of the invention comprises the SPBE, polyolprepolymer-2 and the aforesaid group (A) and (B).

Adapalene is a synthetic retinoid manufactured by Galderma having the following structural formula:

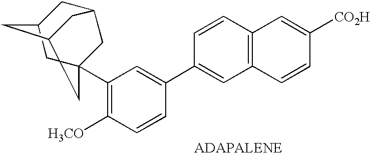

A research article by B. Shroot, “Pharmacodynamics and Pharmacokinetics of Topical Adapalene”, Journal of the American Academy of Dermatology, S17-S24, (1998) has shown that adapalene has several useful properties. First, adapalene has significant comedolytic activity, i.e., it opened comedones or clogged pores. In the preparation of the invention, adapalene acts to enhance the effectiveness of the SPBE. Second, a 0.1 wt. % adapalene gel had a low irritative potential only slightly greater than petroleum jelly used as a control. Third, adapalene has anti-inflammatory effects both in vitro and in vivo. Each of these documented properties provides superiority to the preparations of the invention over the SPBE formulations of the prior art. Lastly, as adapalene is a retinoid, it may be expected to have direct benefits on stimulating hair regrowth.

Adapalene is present in a preparation of the invention in a concentration from about 0.01 wt. % to about 1 wt. %. Preferably, the adapalene concentration is from about 0.025 wt. % to about 0.5 wt. %. Most preferably, the adapalene concentration is about 0.05 wt. %.

Tazarotene is a retinoid having the following structural formula:

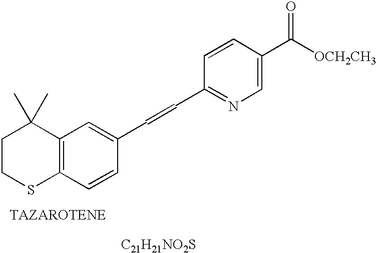

Pharmacologically, tazarotene normalizes keratinocyte differentiation and minimizes proliferation of keratinocytes. These actions serve to inhibit microcomedo formation and prevent follicular plugging. Tazarotene also decreases epidermal inflammation and has been shown to down-regulate biochemical markers of inflammation. In the preparation of the invention, tazarotene acts to enhance the effectiveness of the SPBE.

Tazarotene is present in a preparation of the invention in a concentration from about 0.01 wt. % to about 1 wt. %. Preferably, the tazarotene concentration is from about 0.02 wt. % to about 0.2 wt. %. Most preferably, the tazarotene concentration is about 0.025 to 0.1 wt. %.

Tretinoin has been used in acne therapy. Tretinoin is all-trans retinoic acid, also known as (all E) 3,7-dimethyl-9-(2,6,6-trimethyl-1-cylclohexen-1-yl)-2,4,6,8,-nonatetraenoic acid having the following structural formula:

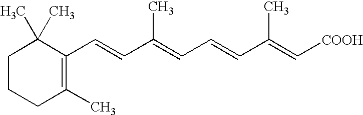

Tretinoin inhibits comedo formation and enhances comedolysis. Thus, it acts to prevent pores from becoming clogged and enhances removal of debris from clogged pores. There is also evidence that tretinoin itself increases hair growth factors. While tretinoin has an irritating potential, it has been found that a 0.025 wt. % tretinoin cream is equivalent in irritation to a 0.1 wt. % adapalene gel. Additionally, a new form of topical tretinoin, RETIN-A MICRO®, has become available from Ortho Dermatological, Raritan N.J. in which 0.1 wt. % of tretinoin is entrapped in a microscopic particle termed a “microsponge”. This particle localizes to the follicle after topical application and then releases tretinoin. The slow release minimizes irritation. By virtue of their increased effectiveness and low irritation potential, a preparation of the invention containing tretinoin in the form of gel microsponges is superior to SPBE preparations of the prior art. Tretinoin is present in a preparation of the invention in a concentration from about 0.005 wt. % to about 0.2 wt. %. Preferably tretinoin concentration is from about 0.025 wt. % to about 0.05 wt. %.

Retinaldehyde is the aldehyde analog of retinoic acid having a terminal aldehyde group in place of the carboxyl group of retinoic acid. It has been shown by J. W. Fluhr et al, Dermatology, 199, Supp 1( ):57-60 (1999) that retinaldehyde is significantly less irritating than retinoic acid (tretinoin). It is expected to be equally as effective in increasing the absorption of SPBE as is tretinoin. Incorporation of retinaldehyde in the SPBE preparations of the invention provides superior effectiveness and lower irritation potential of these preparations over the SPBE formulations of the prior art. Retinaldehyde is present in a preparation of the invention in a concentration from about 0.01 wt. % to about 1.0 wt. %. Preferably the retinaldehyde concentration is from about 0.05 wt. % to about 0.5 wt. %. Most preferably, the retinaldehyde concentration is about 0.1 wt. %.

All trans retinol, or retinol, is a component of numerous over-the-counter skin care products. Like tretinoin, or all trans retinoic acid, topical retinol increases the expression of retinoic acid binding proteins on keratinocytes. It also stimulates hyperplasia of healthy epidermal tissue much the way tretinoin does. Some in fact hypothesize that retinol is a precursor of tretinoin. Further, retinol has been shown to cause less cutaneous irritation than tretinoin. In this invention, retinol is used to enhance the penetration of SPBE into follicle and sebaceous glands. Retinol is present in the invention in a concentration from about 0.01 wt. % to 2.0 wt. %. Preferably, the concentration is between 0.025 wt. % and 1.5 wt. %. Most preferably, the concentration is 1.5 wt. % to 0.5 wt. %.

Alpha hydroxy acids have been shown to exfoliate the stratum comeum. In the present compositions, alpha hydroxy acids act to enhance the effectiveness of SPBE by increasing its penetration into the skin. Glycolic acid, a naturally occurring alpha hydroxy acid, has the formula: HO—CH₂—COOH. Glycolic acid, or another alpha hydroxy acid, could be incorporated into the preparations of the invention. Glycolic acid is present in the present preparations in a concentration from about 0.1 wt. % to about 20 wt. %. Preferably, the glycolic acid concentration is from about 0.5 wt. % to about 10 wt. %. Most preferably, the glycolic acid concentration is about 3 wt. % to 5 wt. %.

Salicylic acid (2-hydroxy benzoic acid) is a beta hydroxy acid having the formula:

OH—C₆H₄—COOH

Beta hydroxy acids are used topically in numerous products to reduce scaling. Salicylic acid, a beta hydroxy acid, is well known as a keratolytic. It increases the penetration of SPBE into follicles and has a low irritancy potential. Incorporation of beta hydroxy acids in general, and salicylic acid in particular, in the SPBE preparations of the invention provides superiority of these preparations over the SPBE formulations of the prior art.

Salicyclic acid is present in the instant preparations in a concentration from about 0.1 wt. % to about 10 wt. %. Preferably, the salicyclic acid concentration is between about 1% wt. % and 2 wt %.

Polyolprepolymer-2 is a urethane compound of molecular weight up to about 200,000 prepared by reacting approximately two moles of a hydroxy terminated linear alkylene or polyalkylene glycol or polyether with approximately one mole of a monomeric organic diisocyanate as described in U.S. Pat. No. 5,700,483 herein incorporated by reference in its entirety. Preferably, polyolprepolymer-2 is of average molecular weight of about 4000 and is prepared by reacting about one mole of dicyclohexylmethanediisocyanate with about two moles of propylene glycol 725.

Incorporation of polyolprepolymer-2 in a topical formulation has been shown to have the characteristic of moderating the rate of transmission of a retinoid to the skin. Specifically, it has been shown that formulations incorporating tretinoin and polyolprepolymer-2 are significantly less irritating and yet therapeutically equally as effective compared to formulations identical except for the absence of the polyolprepolymer-2. When polyolprepolymer-2 is present in a preparation of the invention, tretinoin need not be in the form of gel microsponges. Incorporation of the polyolprepolymer-2 in the SPBE preparations of the invention provides superiority of these preparations over the SPBE formulations of the prior art. The polyolprepolymer-2 is present in a preparation of the invention in a concentration from about 1 wt % to about 20 wt %. Preferably, the polyolprepolymer-2 is present in a concentration from about 2 wt % to about 15 wt %.

The preparations of the invention may include a vehicle for the application to the scalp in the form of a liquid, a gel, a foam, a cleanser or a pad dampened with a liquid. Preferably the SPBE preparations of the invention are employed as a liquid or a gel. Most preferred is a gel.

The formulation of all such topical vehicles is well known to those skilled in the art. A suitable topical vehicle for formulation of the SPBE preparation as a liquid includes ethanol, isopropanol, their mixtures in all proportions. To prepare a liquid preparation of the invention, the SPBE and the penetration enhancing constituents are dissolved or dispersed in the alcohol constituents with agitation. Elevated temperatures may be used to facilitate the dispersion process.

An example of a suitable topical vehicle for formulation of the SPBE preparation as a gel is:

| Component | wt % |
|---|---|
| hydroxypropylcellulose | 2.1 |
| 70/30 isopropyl alcohol/water | 90.9 |
| propylene glycol | 5.1 |
| Polysorbate 80 | 1.9 |

To prepare a gel preparation of the invention, the 70% isopropanol and the propylene glycol are first combined. The SPBE and the penetration enhancing constituents are dispersed in the alcohols with agitation. The hydroxypropylcellulose and the Polysorbate 80 are then incorporated with mixing until a gel results.

An example of a suitable topical vehicle formulation for formulation of the SPBE preparation as a foam is:

| Component | wt % |
|---|---|
| cetyl alcohol | 1.1 |
| stearyl alcohol | 0.5 |
| Quaternium 52 (52%) | 1.0 |
| propylene glycol | 2.0 |
| Ethanol 95 PGF3 | 61.05 |
| deionized water | 30.05 |
| P75 hydrocarbon propellant | 4.30 |

To prepare a foam preparation of the invention, the SPBE and the penetration enhancing constituents are first dispersed in the ethanol at elevated temperature. The cetyl and stearyl alcohols are added to the heated dispersion and mixed until dissolved. The Quaternium 52, the propylene glycol and water are added and stirred until homogeneous while maintaining elevated temperature. The mixture is cooled and dispensed into an aerosol can. A valve is fitted to the can and the can is then charged with the propellant.

The mode of use of a SPBE preparation of the invention is application of 1 cc of the preparation to the affected area of face twice a day for a period of four months. Two cc of the preparation are needed for large areas of the trunk. The preparation should be massaged into the skin or allowed to dry on the skin and remain in place for at least four hours before washing, rinsing or showering. After the four month initial period, a sustaining application of 1 cc once a day is used.

The following examples are presented to provide a more complete understanding of the invention. The specific techniques, conditions, materials, and proportions set forth to illustrate the principles and practice of the invention are exemplary and should not be construed as limiting the scope of the invention.

### EXAMPLES

### Examples 1-14

Tables I and II below provide examples of preparations of the invention. The SPBE in this table is provided by U.S. Nutraceuticals, EUSTICE, Fla. and has a phytosterol concentration of greater than 0.15 wt. %. The topical vehicle may be chosen appropriate to the use of the preparation as a liquid gel, a foam, a cleanser, or a pad dampened with a liquid. All percentages are by weight. B in the tables stands for the Balance of the Composition

TABLE I

| Constituent - % | Ex. 1 | Ex. 2 | Ex. 3 | Ex. 4 | Ex. 5 | Ex. 6 | Ex. 7 |
|---|---|---|---|---|---|---|---|
| SPBE | 31.25 | 31.25 | 31.25 | 31.25 | 31.25 | 31.25 | 31.25 |
| keratinolytics | | | | | | | |
| glycolic acid | 3.0 | | 2.0 | | 2.0 | | 3.0 |
| salicylic acid, | | 1.0 | | 2.0 | | 1.0 | |
| comedolytics | | | | | | | |
| retinol | .1 | | | | | .1 | |
| Retinaldehyde | | .1 | | | | | .05 |
| tretinoin | | | .05 | | | | |
| Adopalene | | | | .025 | | | |
| tazarotene, | | | | | .025 | | |
| Topical Vehicle | B | B | B | B | B | B | B |

TABLE II

| | | | Ex. | Ex. | Ex. | Ex. | Ex. |
|---|---|---|---|---|---|---|---|
| Constituent - % | Ex. 8 | Ex. 9 | 10 | 11 | 12 | 13 | 14 |
| SPBE | 25.0 | 25.0 | 25.0 | 25.0 | 25.0 | 25.0 | 25.0 |
| keratinolytics | | | | | | | |
| glycolic acid | 3.0 | | 2.0 | | 2.0 | | 3.0 |
| salicylic acid | | 1.0 | | 2.0 | | 1.0 | |
| comedolytics | | | | | | | |
| retinol | .1 | | | | | .1 | |
| Retinaldehyde | | .1 | | | | | .05 |
| tretinoin | | | .05 | | | | |
| Adopalene | | | | .025 | | | |
| tazarotene | | | | | .025 | | |
| Polyolprepolymer 2 | 10 | 10 | 10 | 10 | 10 | 10 | 10 |
| Topical Vehicle | B | B | B | B | B | B | B |

The scope of the present invention is set forth in the following claims.

1. A low irritability topical preparation for the treatment of acne and/or hirsutism comprising an effective amount of saw palmetto berry extract containing phytosterols and at least one penetration enhancing constituent each selected from both (A) comedolytic and (B) keratinolytic groups.

2. The topical preparation of claim, 1, wherein the penetration enhancing constituents of group (A) comedolytic is selected from the group consisting of adapalene, tretinoin, tretinoin gel microsponges, retinaldehyde, retinol, and tazarotene and group (B) keratinolytic is selected from the group consisting of beta hydroxy acid and alpha hydroxy acids.

3. The topical preparation of claim 2, wherein the alpha hydroxy acid is glycolic acid.

4. The topical preparation of claim 2, wherein the beta hydroxy acid is salicylic acid.

5. The topical preparation of claim 1, wherein the concentration of saw palmetto berry extract is at least 0.5 wt. %.

6. A topical preparation of claim 1, wherein the preparation is in a topical vehicle selected from the group consisting of a liquid, a dampened pad, a lotion, a cream, a gel, or a foam.

7. The topical preparation of claim 1 wherein said saw palmetto berry extract is obtained by Supercritical CO₂ extraction.

8. A method for the treatment of acne or hirsutism comprising topically applying to the affected area a composition containing an effective amount of saw palmetto berry extract in combination with a penetration enhancing constituent selected from both (A) comedolytic and (B) keratinolytic groups.

9. The method of claim 8, wherein said penetrating enhancing constituents of (A) comedolytic group is selected from the group consisting of adalpalene, tretinoin, tretinoin gel microsponges, retinaldehyde, retinol, and tazarotene, and of the (B) keratinolytic group is selected from beta hydroxy acids and alpha hydroxy acids.

10. The method of claim 8 wherein said keratinolytic group is selected from salicytic acid and glycolic acid.

11. The method of claim 8, wherein said saw palmetto berry extract comprises at least 0.5 wt. % of the topically applied composition.

12. The method of Claim 8 wherein said saw palmetto berry extract is obtained by Supercritical CO₂ extraction.

13. A method for the treatment of acne or hirsutism comprising:
(a) washing the affected area with a mild soap,
(b) applying to the affected area a topical preparation comprising an effective amount of saw palmetto berry extract and penetrating enhancing constituents in accordance with claim 1;
(c) and increasing the frequency of said application if no allergy irritation occurs.

14. The method of claim 13, wherein said topical preparation is applied once daily and thereafter increased to twice daily if no allergy or irritation occurs.

15. The method of claim 13, wherein once a steady state condition has been reached, the frequency of topical application of said composition is reduced to once daily.

16. The method of claim 13, wherein the present topical preparation is applied after mild soap, but before application of moisturizer, sunscreen or makeup.
